(19) United States
(12) Patent Application Publication
(10) Pub. No.: US 2018/0078273 A1
(43) Pub. Date: Mar. 22, 2018

(54) OPTICAL DERMATOME DEPTH GAUGE

(71) Applicant: The Regents of the University of Colorado, a body corporate, Denver, CO (US)

(72) Inventors: Steven L. Moulton, Littleton, CO (US); Maxiliaan Gulden, Winter Park, CO (US); Ram Mahajan, Boulder, CO (US); Tyler Ziebelman, Boulder, CO (US); Erik Mumm, Longmont, CO (US)

(21) Appl. No.: 15/568,771

(22) PCT Filed: Apr. 22, 2016
(86) PCT No.: PCT/US2016/028935

§ 371 Date: Oct. 23, 2017

Related U.S. Application Data
(60) Provisional application No. 62/152,494, filed on Apr. 24, 2015.

(51) Int. Cl. A61B 17/322

(52) U.S. Cl. CPC A61B 17/322; A61B 2090/062

(57) ABSTRACT

Technology is provided for optical dermatome depth gauges. The optical dermatome depth gauge includes a frame and an adjustment mechanism mounted on the frame and including a bore-sight adjustment axis and a height adjustment axis orthogonal to the bore-sight adjustment axis. A dermatome holder is mounted to the adjustment mechanism. A microscope having a microscope axis is mounted to the frame, wherein the microscope axis is parallel to the bore-sight adjustment axis.

### CROSS-REFERENCE TO RELATED APPLICATIONS

This application claims priority to and the benefit of U.S. Provisional Application No. 62/152,494, filed Apr. 24, 2015, the disclosure of which is hereby incorporated by reference in its entirety.

### TECHNICAL FIELD

This patent application is directed to optical depth gauges and, more specifically, to optical dermatome depth gauges.

### BACKGROUND

In the United States, there are over 40,000 hospitalizations per year related to burn injuries. Approximately 26.6% of hospitalized burn patients require skin grafts. Almost all split thickness skin graft harvesting procedures use a dermatome.

A dermatome is an air driven or electrically powered device that is used to harvest split thickness skin grafts from uninjured areas of the body (called donor sites) to provide coverage for an open wound. Split thickness skin grafts are composed of the outermost layers of the skin, including the epidermis and a portion of the underlying dermis. The resulting donor site is essentially a partial thickness burn with residual dermal elements that allow it to heal on its own.

Most split thickness skin grafts are 0.008-0.015 inches (0.20-0.40 mm) thick, and every dermatome has an adjustable gauge to set the desired thickness of the skin graft to be harvested. Unfortunately, dermatomes are rather crude mechanical instruments, and the gauges that are used to set the depth of the cut are somewhat inaccurate and inconsistent. The Zimmer Company reports the accuracy of their dermatome to be +/−0.002 inches. This inaccuracy is especially problematic when caring for burn injured children, because 1) infants and young children have thin skin compared to older children and adults; and 2) the thickness of human skin is variable, depending on the area of the body from which it is harvested.

Traditional mechanisms for determining the thickness of a skin graft are integral to the dermatome itself. It is usually a rotational feature on the side of the dermatome that adjusts a guide up and down relative to the cutting blade of the dermatome. This system is not accurate enough for use on thin skin, such as is found on children. According to the American Burn Association, 30% of burn patients are less than 16 years old. If a skin graft is too thin, it will result in “skipping” and damage to the skin graft, making it unusable. A new donor site will then need to be selected, resulting in multiple donor wounds. On the other hand, if a skin graft is too thick, it will result in a scar at the donor site and longer healing time which correlates to an increase in infection probability. Accordingly, a reliable and consistent method is needed to accurately predetermine the depth that a dermatome will cut before a skin graft is harvested.

### BRIEF DESCRIPTION OF THE DRAWINGS

Embodiments of the optical dermatome depth gauges introduced herein may be better understood by referring to the following Detailed Description in conjunction with the accompanying drawings, in which like reference numerals indicate identical or functionally similar elements.

FIG. 1 is a front view of a dermatome.

FIG. 2 is a schematic illustration of a microscope depth gauge.

FIG. 3 is a perspective view of an optical dermatome depth gauge according to a representative embodiment.

FIG. 4 is a perspective view of an optical dermatome depth gauge according to another representative embodiment.

FIG. 5 is a perspective view of an optical dermatome depth gauge according to a further representative embodiment.

FIG. 6A is an exploded perspective view of the microscope shown in FIG. 5.

FIG. 6B is a representation of a reticle image.

FIG. 7 is a side view in elevation of the frame shown in FIG. 5.

FIG. 8 is an enlarged partial cross-section of a swing stand pivot.

FIG. 9 is a perspective view of a front bracket.

FIG. 10 is a bottom view of the front bracket.

FIG. 11 is a perspective view of the frame side plates.

FIG. 12 is a perspective view of the swing stand.

FIG. 13A is a side view in elevation of the optical dermatome depth gauge with the swing stand in an open position.

FIG. 13B is a side view in elevation of the optical dermatome depth gauge with the swing stand in a collapsed position.

FIG. 14 is a perspective view of an adjustment mechanism.

FIG. 15 is a side view in cross-section of the adjustment mechanism.

FIG. 16 is a perspective view of a dermatome holder.

FIG. 17 is a perspective view of the dermatome holder as viewed from the front with a dermatome mounted thereto.

FIG. 18 is a partial side view of the dermatome holder as viewed from the back.

FIG. 19 is a perspective view of an optical dermatome depth gauge according to a further representative embodiment.

The headings provided herein are for convenience only and do not necessarily affect the scope or meaning of the claimed embodiments. Further, the drawings have not necessarily been drawn to scale. For example, the dimensions of some of the elements in the figures may be expanded or reduced to help improve the understanding of the embodiments. Moreover, while the disclosed technology is amenable to various modifications and alternative forms, specific embodiments have been shown by way of example in the drawings and are described in detail below. The intention, however, is not to limit the embodiments described. On the contrary, the embodiments are intended to cover all modifications, equivalents, and alternatives falling within the scope of the embodiments as defined by the appended claims.

### DETAILED DESCRIPTION

The following description provides specific details for a thorough understanding and enabling description of these examples. One skilled in the relevant art will understand, however, that the techniques discussed herein may be practiced without many of these details. Likewise, one skilled in the relevant art will also understand that the technology can include many other features not described in detail herein. Additionally, some well-known structures or functions may not be shown or described in detail below so as to avoid unnecessarily obscuring the relevant description.

The terminology used below is to be interpreted in its broadest reasonable manner, even though it is being used in conjunction with a detailed description of some specific examples of the embodiments. Indeed, some terms may even be emphasized below; however, any terminology intended to be interpreted in any restricted manner will be overtly and specifically defined as such in this section.

The optical dermatome depth gauges disclosed herein use a unique, no-touch technique to predetermine how thick a skin graft will be prior to harvest. It is not only safe, but it will not harm the dermatome blade and furthermore, is much more accurate and consistent than previous techniques for predetermining graft thickness. For example, in the past, a surgeon might set the gauge on the side of the dermatome and then insert a scalpel of a known thickness into the gap in an effort to check the gap size. This technique has the potential to damage the dermatome blade and is not a reliable gauge of graft thickness.

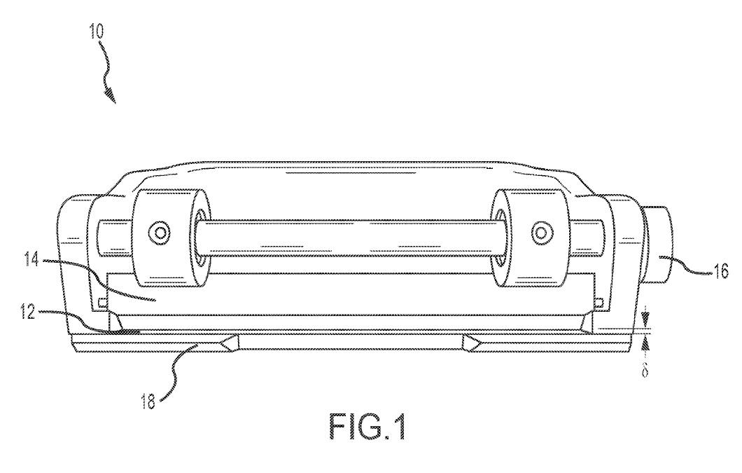

The optical dermatome depth gauges described herein accurately predetermine the thickness of a skin graft that a dermatome will harvest. With reference to FIG. 1, the thickness of skin harvested (δ) corresponds to the gap between the blade 12 and the guide 14 of the dermatome 10. The guide 14 moves vertically up and down as the side knob 16 for thickness adjustment on the dermatome 10 is rotated. This alters the gap width (δ), thus resulting in harvesting skin grafts of variable thickness. The objective is to accurately measure the skin graft thickness (δ), which translates into being able to accurately measure the gap (δ) between the guide 14 and blade 12. The width plate 18 determines how wide the graft will be.

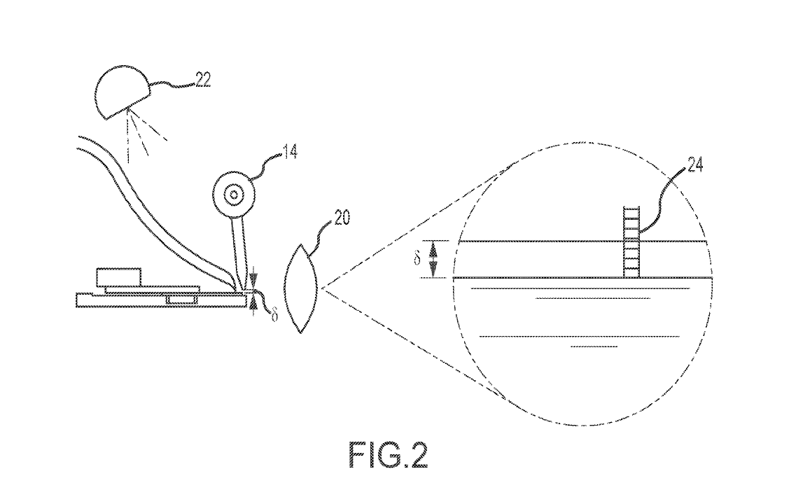

As shown in FIG. 2, the gap (δ) is measured using a magnifying lens 20 (e.g., microscope or other magnification device). The gap (δ) is illuminated by a light source 22 from behind the guide 14. The magnifying lens 20 magnifies the gap (δ), which makes it discernible to the viewer. A reticle 24 in the magnifying lens 20 provides a scale of known length to enable the viewer to make the measurement.

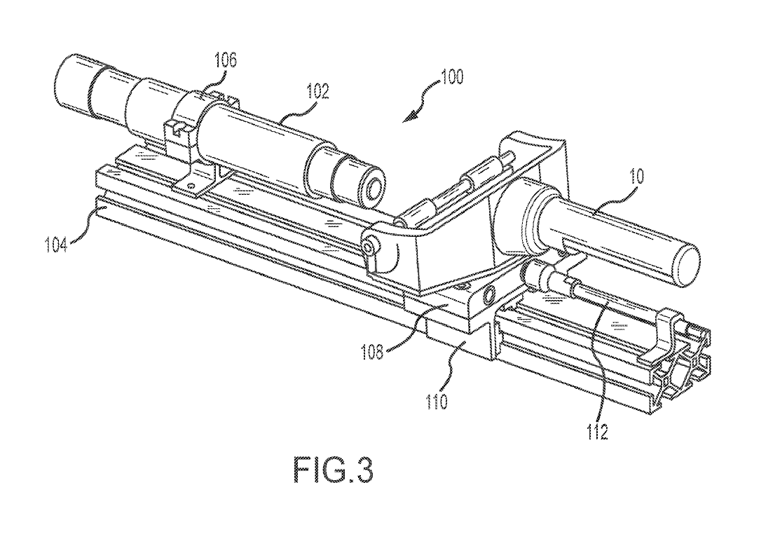

FIG. 3 illustrates an optical dermatome depth gauge 100 according to a representative embodiment. The optical dermatome depth gauge 100 includes an extruded rail 104 that provides adjustment options for fine tuning of the system. The modular nature of this design also makes it convenient to quickly adjust. Optical dermatome depth gauge 100 also includes microscope clamp 106 for mounting the microscope 102. The optical dermatome depth gauge 100 includes a rail sled 108 with adjustment rod 112 and vertical guide 110.

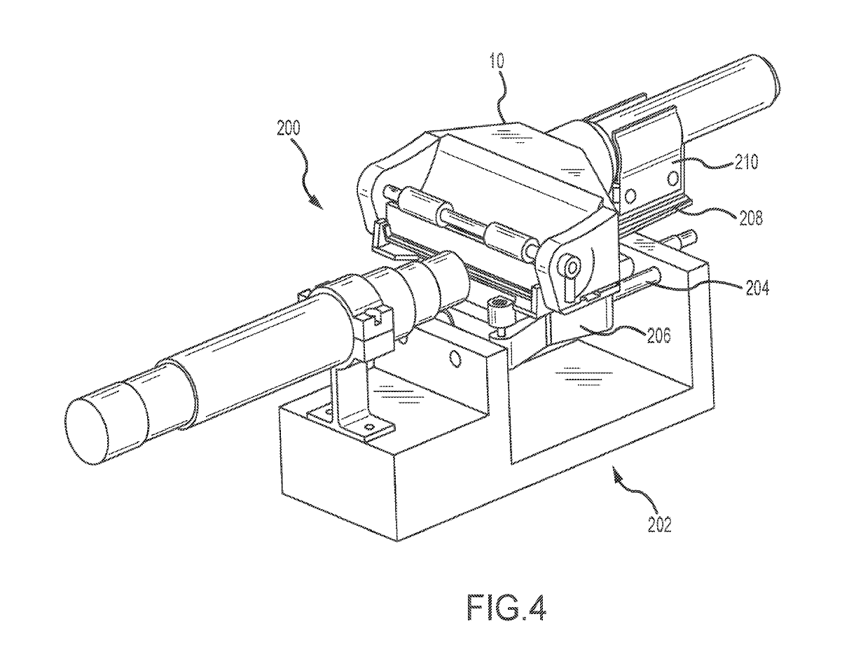

FIG. 4 illustrates an optical dermatome depth gauge 200 according to another representative embodiment. Optical dermatome depth gauge 200 incorporates a solid block base 202 that fixes all length aspects. Guide rails 204 are inserted into the solid block base 202, which determines the adjustment axes. A focus and height adjustment mechanism 206 resides between the guide rails 204 and provides both focus and height adjustments. The adjustment mechanism 206 supports the base plate 208 of the dermatome holder on which the bracket 210 of the dermatome holder is attached via a suitable fastener, such as a screw.

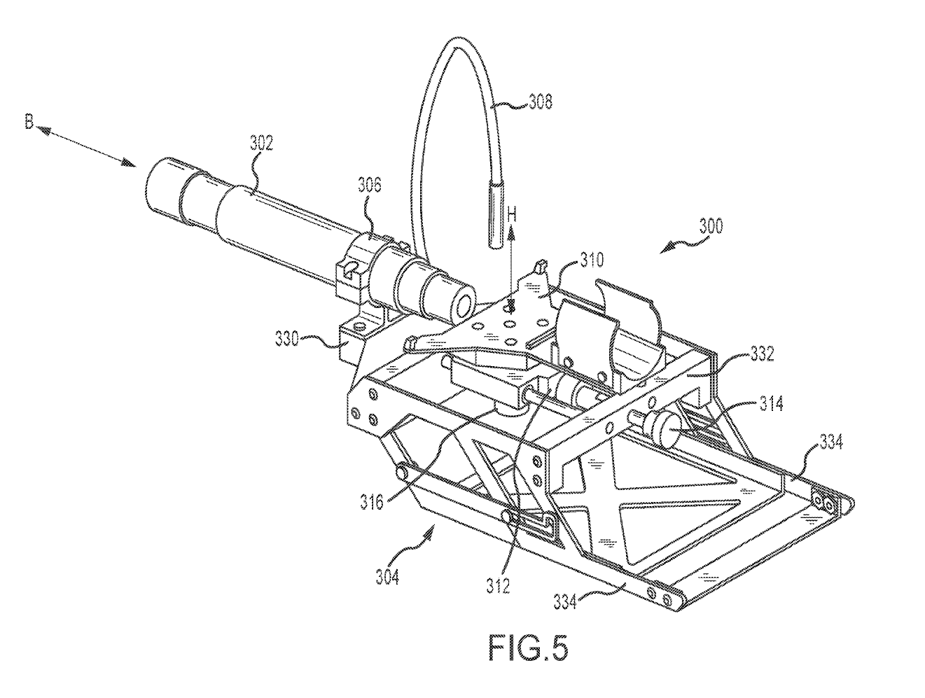

FIG. 5 illustrates an optical dermatome depth gauge 300 according to a further representative embodiment. Optical dermatome depth gauge 300 includes a frame 304 upon which is mounted a microscope 302. Frame 304 includes front bracket 330 and rear bracket 332 mounted between side plates 334. Microscope 302 is mounted to the frame 304 with clamp assembly 306. Light source 308 is attached to the frame 304 and provides illumination for measuring the gap. In some embodiments, the light source 308 is a white light source, which is suitable for all types of objective lenses. The gooseneck design can be adjusted to point at the gap. Dermatome holder 310 is mounted to an adjustment mechanism 312 that is in turn mounted to frame 304. The frame 304 and alignment mechanism 312 may be broadly referred to as an alignment structure. The adjustment mechanism 312 provides bore adjustment B via knob 314 for moving the dermatome along the microscope's bore-sight axis (i.e., microscope axis) in order to focus the microscope on the dermatome blade-guide gap. The adjustment mechanism 312 provides height adjustment H via knob 316 for moving the dermatome up and down with respect to the bore-sight axis of the microscope 302, thereby allowing the blade of the dermatome to be aligned with the microscope reticle. In some embodiments, the microscope can be moved relative to the dermatome rather than moving the dermatome relative to the microscope.

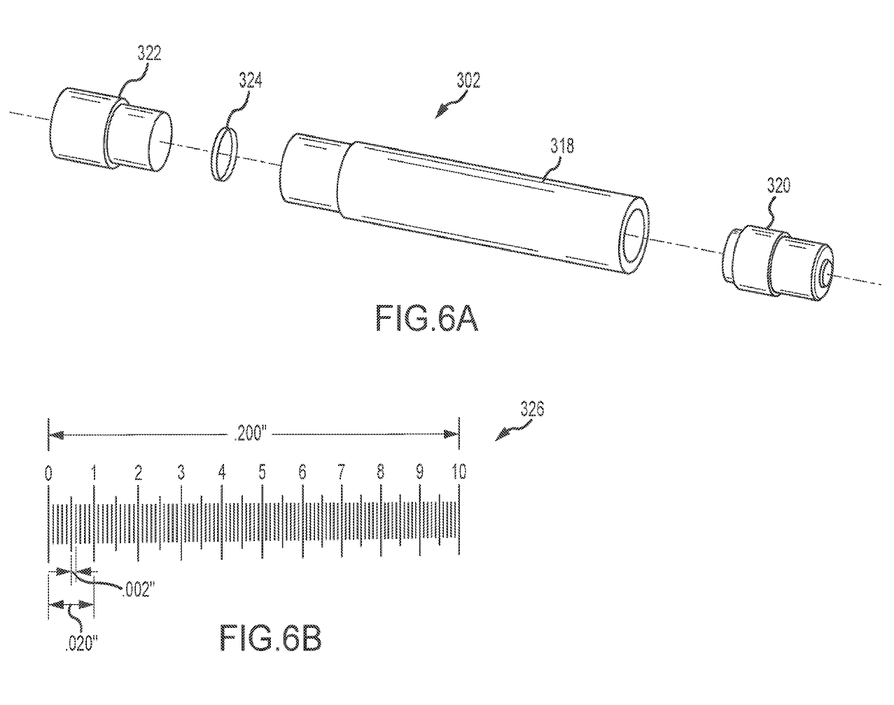

As shown in FIG. 6A, the microscope 302 includes a lens tube 318, an objective 320, an eyepiece 322, and a reticle 324. The objective 320 is the lens closest to the object being observed. In some embodiments, the objective has a magnification of 10× and has a working distance of 10 mm. The eyepiece 322 is the lens closest to the observer. In some embodiments, the eyepiece 322 has a magnification of 10×. The eyepiece 322 also has an integral minor focus adjustment and houses the reticle 324. The reticle 324 is a lens with a scale 326 (see FIG. 6B) and offers no magnification. In some embodiments, the scale 326 is 0.2″ in length and has 100 divisions as seen in FIG. 6B. In some embodiments, the reticle 324 fits inside the eyepiece 322. The lens tube 318 connects the objective 320 to the eyepiece 322. The image magnified by the objective 320 lies on the focal plane of the eyepiece 322, which puts a restriction on the distance between the eyepiece 322 and the objective 320. This distance depends upon the standard the lens must adhere to, which, in this case, is the DIN standard (German optical standards). The selected lens tube adheres to the DIN standard and keeps lenses at the specified distance.

The objective 320 magnifies the gap 10× and projects the image on the focal plane of the eyepiece 322. This focal plane coincides with the plane at which the reticle 324 is placed. The image of the gap is magnified by 10× in this plane and is superimposed on the reticle 324. The eyepiece 322 then magnifies the reticle 324 and the magnified gap from the objective 320 by an additional 10×. Thus, the observer perceives the gap magnified by 100× and the reticle magnified by 10×.

TABLE 1

| | | | |
|---|---|---|---|
| Objective | 10 | | |
| magnification | | | |
| Eyepiece | 10 | | |
| magnification | | | |
| Reticle length | 0.2 | | |
| (inches) | | | |
| Reticle divisions | 100 | | |
| Actual gap width | 0.002 | Corresponding image length | 0.2 |
| corresponding to 10 | | after magnification (inches) | |
| reticle divisions | | | |
| (inches) | | | |
| Actual gap width | 0.0002 | Corresponding image length | 0.02 |
| corresponding to 1 | | after magnification (inches) | |
| reticle divisions | | | |
| (inches) | | | |
| No. of reticle divisions | 50 | Corresponding image length | 1.2 |
| for 12/1000″\| | | after magnification (inches) | |
| No. of reticle divisions | 5 | Corresponding image length | 0.1 |
| for 1/1000″ | | after magnification (inches) | |
| No. of reticle divisions | 2.5 | Corresponding image length | 0.05 |
| for 5/10000″ | | after magnification (inches) | |

The example objective 320 and eyepiece 322 magnification selections above provide the ability to measure from 0.001″ to 0.016″. Out of the 100 divisions of the reticle 324, the 60th division corresponds to 0.012″ as seen from Table 1. Thus, the required range of measurement fits on the reticle 324 and allows measurement up to 0.020″. The accuracy of 0.0005″ corresponds to 2.5 divisions on the reticle 324 as shown in Table 1. The minimum distance one can measure using this microscope configuration is 0.0002″. This exceeds the desired accuracy of 0.0005″, and thus, both the range and accuracy requirements have been met.

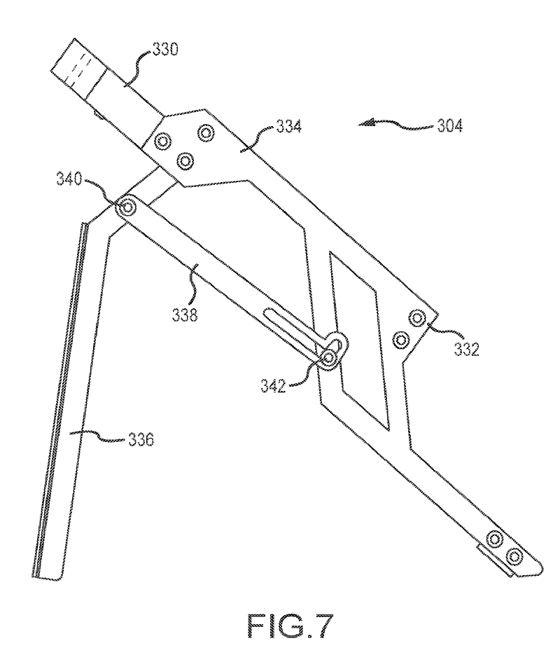

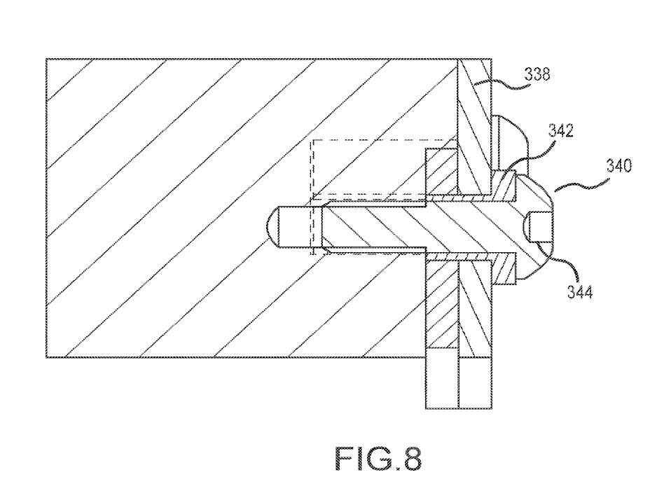

With reference to FIG. 7, the frame 304 is designed to provide structure and stability for the entire mechanism. The frame 304 is designed to provide a 45° viewing angle via a collapsible swing stand 336 and locking arms 338 located on the sides to prevent it from accidentally collapsing. An adjustment may be made to increase the viewing angle. In some embodiments, the frame pieces are made using 1/16″ sheet aluminum water jet cut to the required shapes. The locking arms 338 connect the swing stand 336 to the side plates 334 via a fixed hinge 340 and a slider 342. As the swing stand 336 extends, each locking arm 338 slides over the slider 342, and at the end of the extension, the locking arm 338 drops and locks the system until physically lifted out of the locking position. As shown in FIG. 8, the fixed hinge 340 includes a bushing 342 disposed between the fastener 344 and locking arm 338. In some embodiments, the bushing 342 can be comprised of ultra-high molecular weight polyethylene (UHMWPE).

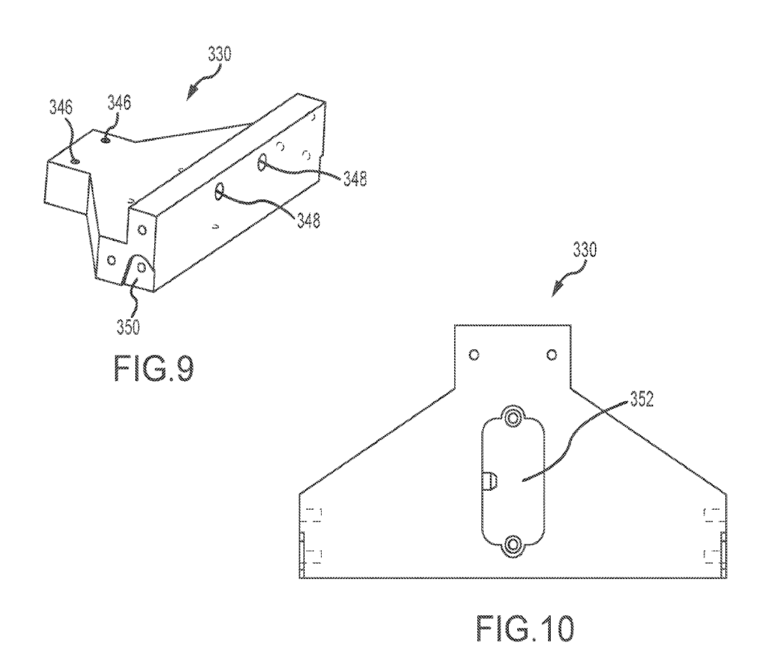

The front bracket 330 and the rear bracket 332 are mounted between the side plates 334 and provide mounting support for the adjustment mechanism 312 (see FIG. 5). Also, as shown in FIGS. 9 and 10, front bracket 330 includes a battery enclosure 352 which houses a 12 V battery required for the light source 308. The light source 308 is also attached to the front bracket 330. This puts the light in the optimal location to be positioned over the dermatome 10. The microscope clamp assembly 306 is also attached to the front bracket 330 via mounting apertures 346. The front bracket 330 also includes swing stand hinge points 350. These features keep the swing stand 336 in the proper range of motion by limiting its rotation. Apertures 348 provide mounting locations for the adjustment mechanism 312.

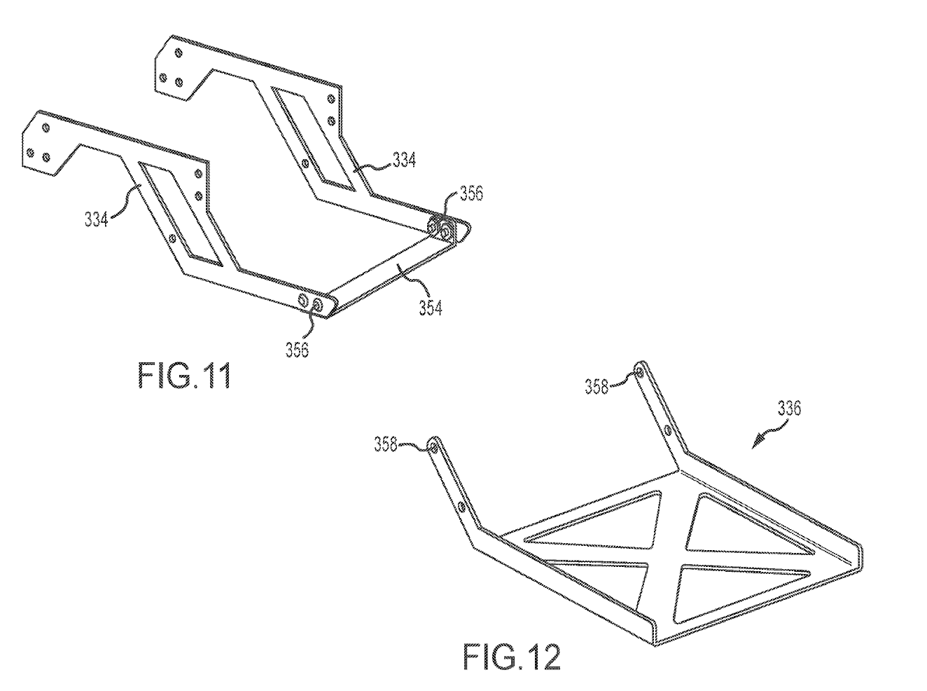

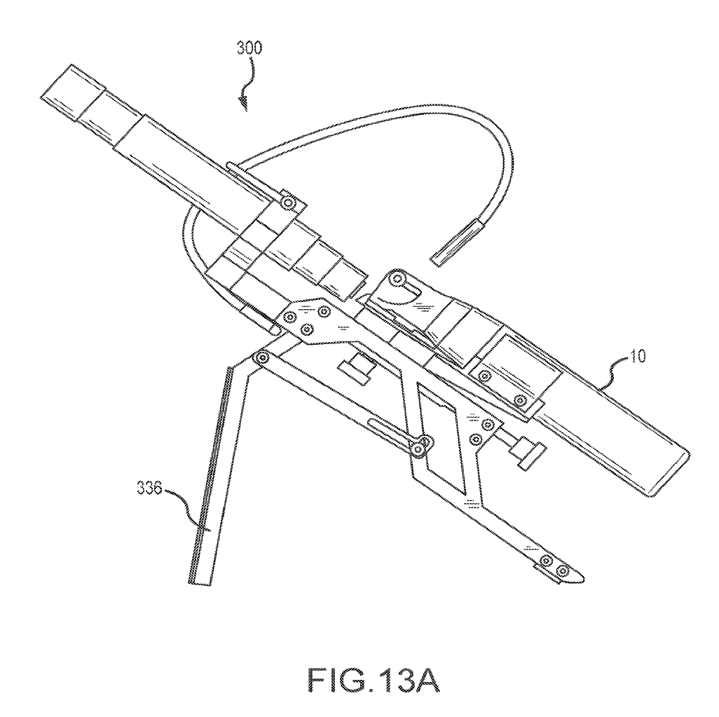

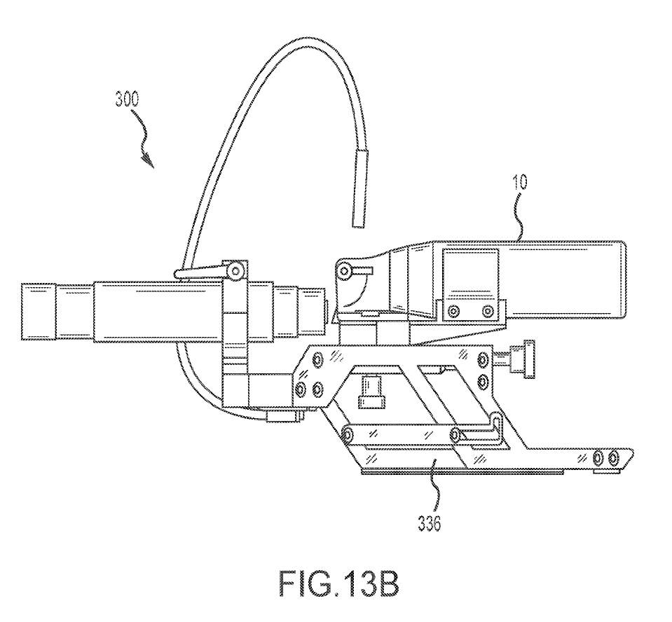

As shown in FIG. 11, the side plates 334 provide the initial structure, spacing and pivot points for the swing stand 336. However, the tail is stabilized by a cross-member bracket 354 to prevent flex. In some embodiments, the cross-member bracket 354 is held in place by suitable fasteners 356 to provide rigidity. As shown in FIG. 12, the swing stand 336 can be made from a single sheet of aluminum folded to maintain rigidity. The swing stand 336 has been designed to provide a 60° swing and a large structural base. The hinge points 358 for the swing stand 336 are aided with UHMWPE bushings (see FIG. 8) to provide a low friction surface. These hinge points 358 are located between the side plates 334 and front bracket 330, so when everything is bolted together, the hinge points are locked into the frame. FIGS. 13A and 13B illustrate the swing stand 336 in the open position and collapsed position, respectively.

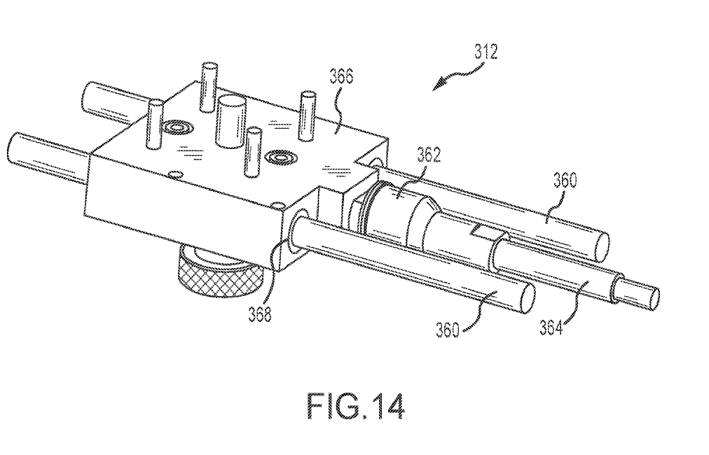

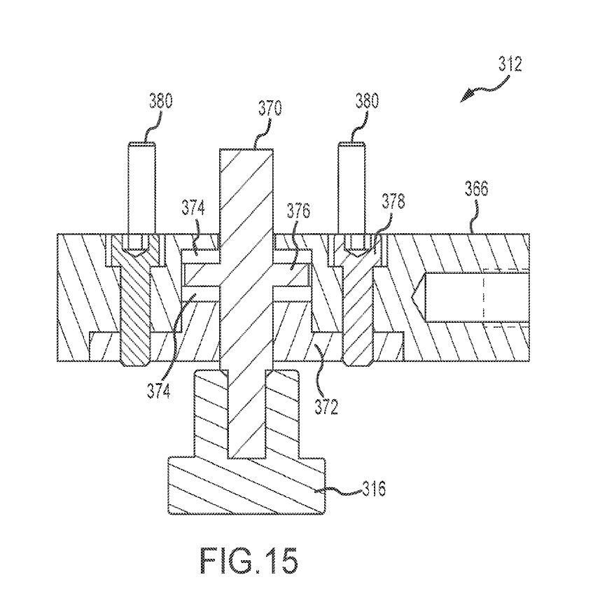

The adjustment mechanism 312, shown in FIGS. 14 and 15, allows the dermatome 10 to be adjusted along the bore-sight (focus) and perpendicular to the microscope. The perpendicular (height) adjustment allows the blade of the dermatome to be aligned properly with the microscope reticle, and the bore-sight adjustment provides the major focusing movement for the microscope. The adjustment mechanism 312 also keeps the microscope 302 in line with the dermatome 10 through appropriate tolerances on the parts. The adjustment mechanism 312 includes a pair of horizontal rails 360 that span between the front bracket 330 and rear bracket 332 as shown in FIG. 5. The horizontal rails 360 can be bearing-quality alloy steel and purchased from McMaster-Carr. In some embodiments, these horizontal rails are press fit into the front bracket and rear bracket.

The adjustment mechanism 312 incorporates both the bore-sight and height adjustments. With reference to FIG. 14, the bore-sight direction is adjusted with a ball and socket joint 362 attached to a ¼″-28 threaded rod 364 to move the platform 366 in the bore-sight direction. Threaded rod 364 mates with threads formed in the rear bracket 332. This threading results in 0.035″ of bore-sight movement per revolution, which is used as the major focusing movement for the microscope 302. To facilitate the bore-sight movement, four UHMWPE bushings 368 are inserted in the holes for the horizontal rails 360.

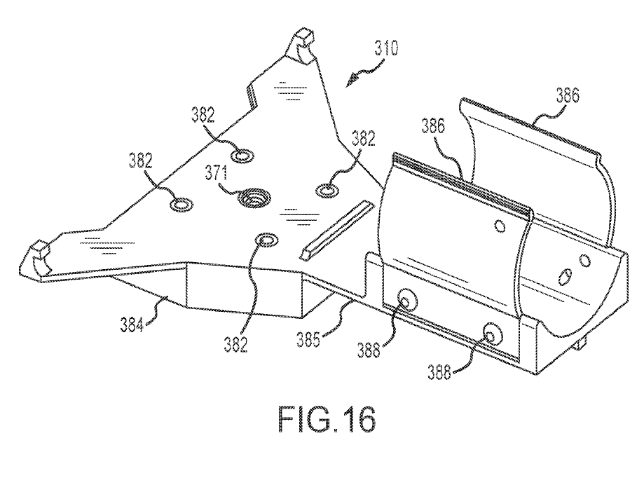

As shown in FIG. 15, the height adjustment is provided by a rotary shaft 370 that is rotatably mounted in the platform 366. Rotary shaft 370 includes a disc portion 376 that is sandwiched between a pair of thrust bearings 374. In some embodiments, the thrust bearings are comprised of UHMWPE. The rotary shaft 370 and thrust bearings 374 are clamped in the platform 366 by retainer 372 that is attached to the platform 366 by suitable fasteners 378. The end of rotary shaft 370 includes ¼″-20 threads that mate with corresponding threads 371 (see FIG. 16) in the dermatome holder 310. Thus, when the rotary shaft 370 is rotated, the dermatome holder 310 moves up or down according the thread pitch and the number of rotations of the rotary shaft 370. These threads result in 0.05″ of height movement per revolution. A plurality of alignment pins 380 are mounted on the platform 366 and mate with corresponding guide bushings 382 (see FIG. 16) in the dermatome holder 310. With reference to FIG. 16, these alignment pins 380 slide in and out of the guide bushings 382 and keep the dermatome 10 aligned with the microscope 302.

As shown in FIG. 16, the dermatome holder 310 includes a base portion 384 and a cradle portion 385 with a pair of handle clips 386 attached to the cradle portion 385 with suitable fasteners 388. In this embodiment, the cradle portion 385 is rounded to match the handle of the dermatome. In some embodiments, the handle clips 386 are comprised of titanium brackets, each secured by 2× #6-32 button head screws into the cradle portion 385. The handle clips 386 are configured to wrap around the dermatome handle securing it to the cradle portion. Although, the design described herein is specific to a particular dermatome (e.g., Zimmer Air Dermatome II), the platform 366 has also been designed so that the dermatome holder 310 can be removed from the adjustment mechanism 312 to allow interchanging of dermatome holder models. In some embodiments, the dermatome holder 310 can comprise plastic or metal, such as, for example, ABS plastic or aluminum. In some embodiments, corresponding threads 371 are in the form of a ¼″-20 internal thread plastic insert pressed into the center of the base portion 384. In some embodiments, the guide bushings 382 are located in the corners around the central corresponding thread 371 and comprise UHMWPE ⅛″ ID (Close fit), 0.2″ OD (press fit) bushings. They provide a low friction surface for the alignment pins 380 to hold the dermatome level while it is being raised and lowered.

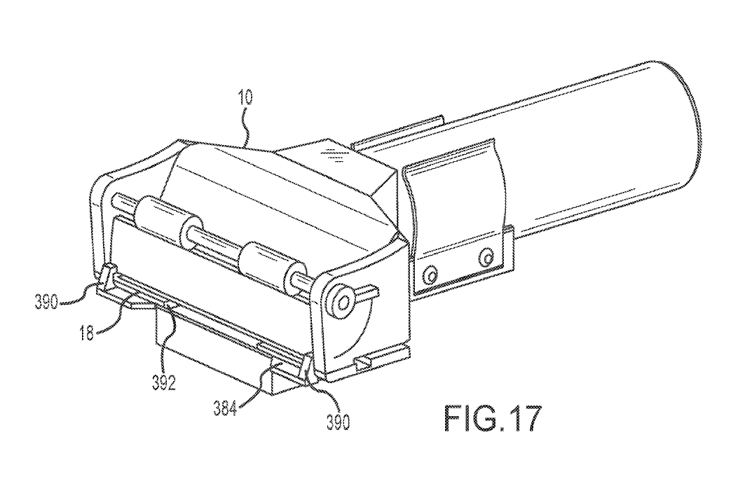

As shown in FIG. 17, located at the front of the dermatome 10 are small front hooks 390 extending from the base portion 384 that lock onto the front edge 392 of the width plate 18 (see FIG. 1). In some embodiments, the dermatome 10 includes small protrusions designed to prevent anything from getting caught in the corners. In this case, the small front hooks 390 lock onto the protrusions. In any case, the small front hooks 390 prevent the front of dermatome 10 from lifting up or sliding forward.

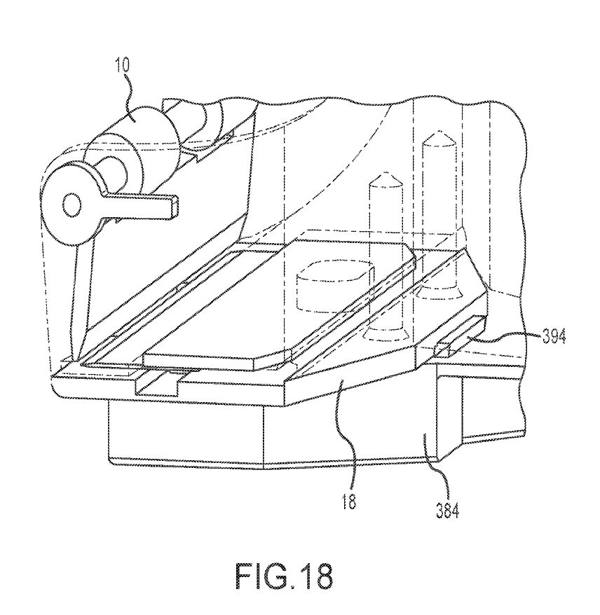

As shown in FIG. 18, at the rear of the dermatome 10 is a ledge 394. This small protrusion in the base portion 384 wedges behind the width plate 18 to prevent the dermatome 10 from sliding back. The leading edge of ledge 394 can also be rounded to ease insertion of the dermatome.

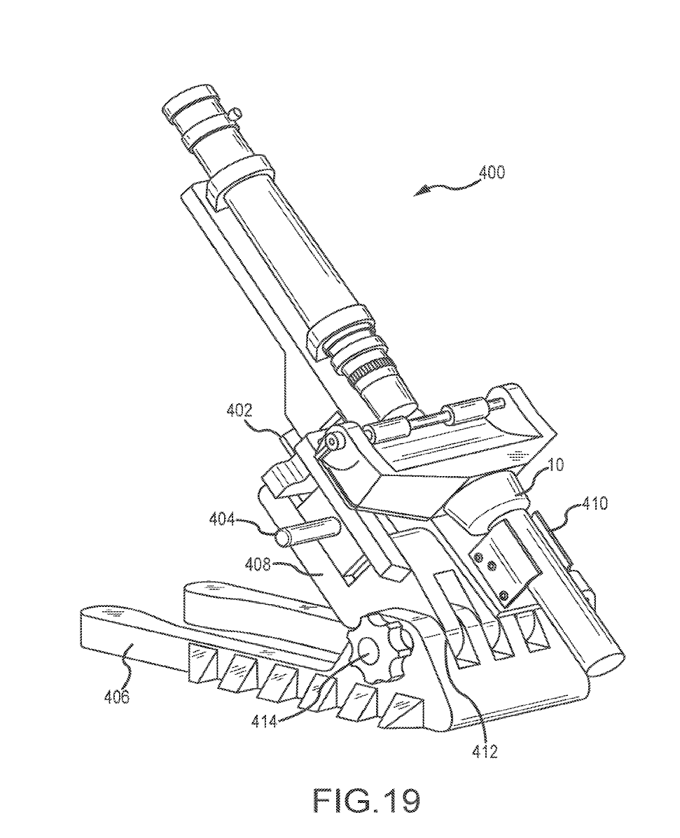

FIG. 19 illustrates an optical dermatome depth gauge 400 according to a further representative embodiment. This embodiment employs concepts similar to those described above. However, in this case, linear guide rails and single point vertical thread are replaced with linear and vertical stages 402, 404 to increase in-use stability. The swing stand is replaced with a support stand comprising a horseshoe base 406 and single pivot arm 408 to reduce the number of components and improve ease of access to the motion dials. The dermatome holder 410 is bolted to the vertical stage 404 via a single bolt, further improving the rigidity of the system. Clutch style hinge 412 provides multiple viewing angles at 5 deg. increments, engaged by knobs 414 located on either side. In some embodiments, the base and/or arm can comprise a plastic material, such as UHMWPE.

Methods relating to the above described optical dermatome depth gauges are also contemplated. The methods thus at least encompass the steps inherent in the above described mechanical structures and operation thereof. Broadly, one representative method of predetermining the thickness of a skin graft includes positioning a dermatome having a blade and a blade guide on an alignment structure and positioning a magnification device having a magnification axis on the alignment structure adjacent the blade. The gap between the blade and blade guide is observed (i.e., measured) and the dermatome is removed from the alignment structure for harvesting a graft. In some embodiments, the magnification device is a microscope including an objective lens and an eyepiece. In some embodiments the method includes positioning a reticle adjacent the eyepiece and comparing the gap between the blade and blade guide to the reticle. In some embodiments, the method includes projecting light from a light source onto the dermatome. In other embodiments, the method includes moving the magnification device in a direction parallel to the magnification device axis and/or moving the magnification device in a direction transverse to the magnification device axis.

The above description and drawings are illustrative and are not to be construed as limiting. Numerous specific details are described to provide a thorough understanding of the disclosure. However, in some instances, well-known details are not described in order to avoid obscuring the description. Further, various modifications may be made without deviating from the scope of the embodiments. Accordingly, the embodiments are not limited except as by the appended claims.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. It will be appreciated that the same thing can be said in more than one way. Consequently, alternative language and synonyms may be used for any one or more of the terms discussed herein, and any special significance is not to be placed upon whether or not a term is elaborated or discussed herein. Synonyms for some terms are provided. A recital of one or more synonyms does not exclude the use of other synonyms. The use of examples anywhere in this specification, including examples of any term discussed herein, is illustrative only and is not intended to further limit the scope and meaning of the disclosure or of any exemplified term. Likewise, the disclosure is not limited to various embodiments given in this specification. Unless otherwise defined, all technical and scientific terms used herein have the same meaning as commonly understood by one of ordinary skill in the art to which this disclosure pertains. In the case of conflict, the present document, including definitions, will control.

1. An optical dermatome depth gauge, comprising:
a frame;
an adjustment mechanism mounted on the frame and including a bore-sight adjustment axis and a height adjustment axis orthogonal to the bore-sight adjustment axis;
a dermatome holder mounted to the adjustment mechanism; and
a microscope having a microscope axis mounted to the frame wherein the microscope axis is parallel to the bore-sight adjustment axis.

2. The optical dermatome depth gauge of claim 1, further comprising a light source mounted to the frame.

3. The optical dermatome depth gauge of claim 1, wherein the microscope includes a reticle.

4. The optical dermatome depth gauge of claim 1, wherein the frame includes a pair of side plates with a front bracket and a rear bracket extending therebetween.

5. The optical dermatome depth gauge of claim 4, wherein the adjustment mechanism includes a pair of guide rails parallel to the bore-sight adjustment axis and extending between the front and rear brackets.

6. An optical dermatome depth gauge, comprising:
a support stand, including:
a base; and
an arm pivotably coupled to the base;
an adjustment mechanism mounted on the arm and including a sight adjustment axis and a height adjustment axis orthogonal to the sight adjustment axis;
a dermatome holder mounted to the adjustment mechanism; and
a magnification device having a device axis mounted to the arm wherein the device axis is parallel to the sight adjustment axis.

7. The optical dermatome depth gauge of claim 6, wherein the adjustment mechanism comprises a linear stage corresponding to the sight adjustment axis and a vertical stage corresponding the to the height adjustment axis.

8. The optical dermatome depth gauge of claim 6, wherein the arm is pivotably coupled to the base with a hinge.

9. The optical dermatome depth gauge of claim 8, further comprising one or more knobs coupled to the hinge, whereby a user can selectively position the arm at a desired angle with respect to the base.

10. The optical dermatome depth gauge of claim 6, wherein at least one of the base and arm comprise a plastic material.

11. The optical dermatome depth gauge of claim 6, wherein the magnification device is a microscope including an objective lens and an eyepiece.

12. The optical dermatome depth gauge of claim 6, further comprising a light source mounted to the support stand.

13. A method for predetermining thickness of a skin graft, the method comprising:
positioning a dermatome having a blade and a blade guide on an alignment structure;
positioning a magnification device having a device axis on the alignment structure adjacent the blade;
observing a gap between the blade and blade guide;
removing the dermatome from the alignment structure.

14. The method of claim 13, wherein the magnification device is a microscope including an objective lens and an eyepiece.

15. The method of claim 14, further comprising positioning a reticle adjacent the eyepiece.

16. The method of claim 15, further comprising comparing the gap between the blade and blade guide to the reticle.

17. The method of claim 13, further comprising projecting light from a light source onto the dermatome.

18. The method of claim 13, further comprising moving the magnification device in a direction parallel to the device axis.

19. The method of claim 13, further comprising moving the magnification device in a direction transverse to the device axis.

20. The method of claim 13, further comprising moving the dermatome in a direction parallel to the device axis.
